(19) United States
(12) Patent Application Publication
(10) Pub. No.: US 2024/0350325 A1
(43) Pub. Date: Oct. 24, 2024

(54) Elastic Laminate

(71) Applicant: APLIX, Le Cellier (FR)

(72) Inventor: Emilie Alexandra Pepion, Le Cellier (FR)

(21) Appl. No.: 18/687,501

(22) PCT Filed: Oct. 17, 2022
(86) PCT No.: PCT/EP2022/078878

§ 371 Date: Feb. 28, 2024

(30) Foreign Application Priority Data
Oct. 19, 2021 (FR) 2111080

(51) Int. Cl. A61F 13/49; A61F 13/56; A61F 13/62

(52) U.S. Cl. CPC A61F 13/49015; A61F 13/5638; A61F 13/5655; A61F 13/622

(57) ABSTRACT

A laminate extending widthwise, in the direction CD, and lengthwise, in the direction MD, including at least one nonwoven layer extending over a total nonwoven width, in the direction CD, and at least one elastic film made of elastomer material fixed by an upper face or lower face, respectively, to a lower face or upper face, respectively, of the at least one nonwoven layer, the at least one elastic film extending over a total elastic width, characterised in that the ratio equal to the total elastic width to the total nonwoven width is between 0.3 and 0.9; and the SET at 100% elongation of the laminate and/or of the at least one elastic film, measured in the width direction, is greater than 6%, in particular between 6% and 30%.

The present invention relates to an elastic laminate, intended to be used in the hygiene field, and particularly for nappies or adult incontinence pants, said laminate extending widthwise, corresponding broadly to the direction CD (cross direction or transverse direction), and lengthwise, corresponding broadly to the unwinding direction during manufacture of the laminate, referred to as the direction MD (machine direction), and comprising a stack of one or more layers of nonwoven and one or more elastic films extending over a width less than or equal to the said one specified width, particularly two layers of lower and upper nonwoven sandwiching the elastic film. These laminates are particularly intended for use in the manufacture of elastic lugs intended to carry hooks and to be fixed to the edges of the rear central part of the waist of a nappy to engage with the loop elements originating from the front part of the waist to create a movable and elastically adjustable closure for the nappy.

A laminate of this type is known for example from document EP-A-1783257, in the name of the applicant.

It is desirable to yet further improve the usage comfort of nappies which comprise lugs formed from an elastic laminate of this kind. In particular, it is desirable for the waist of the nappy, which comprises lugs formed from an elastic laminate of this kind, to adjust better to the shape of the wearer of the nappy.

The present invention thus relates, according to a first aspect, to a laminate extending widthwise, in particular in the direction CD, and lengthwise, particularly in the direction MD, comprising:
at least one nonwoven layer, said nonwoven layer or layers forming the at least one nonwoven layer extending over a total nonwoven width, in particular in the direction CD, and
at least one elastic film made of an elastomer material fixed by an upper face or lower face, respectively, to a lower face or upper face, respectively, of the at least one nonwoven layer, the elastic film or films made of elastomer material forming the at least one elastic film made of elastomer material extending over a total elastic width,
characterised in that
the ratio equal to the total elastic width over the total nonwoven width is between 0.3 and 0.9, preferably between 0.4 and 0.8, in particular between 0.6 and 0.8; and
the SET at 100% elongation of the laminate and/or of the at least one elastic film made of elastomer material, measured, in the width direction, with a sample having a length and a width and wherein the elastomer material extends along the entire length and/or across the entire width of the sample, is greater than 6%, in particular greater than 15%, in particular between 6% and 30%, in particular between 15% and 30%.

Preferably, the SET at 100% of the laminate and/or of the at least one elastic film made of elastomer material is greater than 6%, in particular greater than 7%, 8%, 9%, 10%, 11%, in some cases less than 30%, in particular less than 25%, 20%, 19%, 18%, 17%.

Preferably, according to another aspect of the invention itself forming an invention, independently of the other aspects of the invention, and in particular independently of the first aspect, but which can also be implemented in a favourable manner in combination with each of these other aspects, and in particular with the first aspect, the elastomer material of the elastic film or films has a Shore A hardness, in particular measured according to standard ISO 868:2003, of between 50 and 90, in particular between 60 and 80.

Preferably, according to another aspect of the invention that itself forms an invention, alternatively or in combination with the SET values at 100% of the first aspect of the invention, the SET at 40% elongation of the laminate and/or of the elastic film made of elastomer material, measured with a sample having a length and a width and wherein the elastic made of elastomer material, forming the at least one elastic film made of elastomer material extending over a total elastic width, extends along the entire length and/or across the entire width, is greater than 2.25%, in particular greater than 2.5%, 2.75%, 3%, 3.25%, in some cases less than 15%, in particular less than 14%, 13%, 12%, 11%, 10%, 9%, 8%, 7%, 6%.

Preferably, according to another aspect of the invention that itself forms an invention, alternatively or in combination with the SET values at 100% and 40% of the first aspect of the invention, the SET at 60% elongation of the laminate and/or of the elastic film made of elastomer material, measured with a sample having a length and a width and wherein the elastic extends along the entire length and/or across the entire width, is greater than 3.5%, more particularly greater than 4%, in particular greater than 4.5%, 5%, 5.5%, 6%, 6.5%, 7%, in some cases less than 20%, in particular less than 15%, 14%, 13%, 12%, 11%, 10%, 9%.

Preferably, according to another aspect of the invention that itself forms an invention, alternatively or in combination with the SET values at 100%, 40% and 60% of the first aspect of the invention, the SET at 80% elongation of the laminate and/or of the elastic film made of elastomer material, measured with a sample having a length and a width and wherein the elastic extends along the entire length and/or across the entire width, is greater than 5%, in particular greater than 6%, 7%, 8%, 9%, 10%, in some cases less than 25%, in particular less than 20%, 19%, 18%, 17%, 16%, 15%, 14%, 13%.

Preferably, according to another aspect of the invention that itself forms an invention, alternatively or in combination with the SET values at 100%, 40%, 60% and 80% of the first aspect of the invention, the SET at 120% elongation of the laminate and/or of the elastic film made of elastomer material, measured with a sample having a length and a width and wherein the elastic extends along the entire length and/or across the entire width, is greater than 7%, in particular greater than 8%, 9%, 10%, 11%, 12%, 13%, 14%, 15%, 16%, 17%, 18%, 19%, 20%, in some cases less than 30%, in particular less than 29%, 28%, 27%, 26%, 25%, 24%.

Preferably, at least one of the values of the ratio [SET (as a %)/elongation (as a %/100)] of the laminate,
if breaking of the laminate occurs beyond the elongation to 120%, measured for the elongations to 40%, 60%, 80%, 100% and 120%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120%, a return to 0%, a sixth elongation to 40%, a return to 0%, and an elongation to break, the SET of the elongations to 40%, 60%, 80%, 100% and 120% being calculated, respectively, at the second, third, fourth, fifth and sixth elongation to 40%, or
if breaking of the laminate occurs at an elongation of between 100% and 120%, measured for the elongations to 40%, 60%, 80% and 100%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, and an elongation to break, the SET of the elongations to 40%, 60%, 80% and 100% being calculated, respectively, at the second, third, fourth and fifth elongation to 40%, or
if breaking of the laminate occurs at an elongation of between 80% and 100%, measured for the elongations to 40%, 60% and 80%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, and an elongation to break, the SET of the elongations to 40%, 60% and 80% being calculated, respectively, at the second, third and fourth elongation to 40%, is between 6 and 30, in particular between 8 and 25, in particular between 8 and 20, the elongation being carried out according to a direction in CD (cross direction) or in MD (machine direction).

In particular, for at least two of the elongations to 40%, 60%, 80%, 100% and 120%, in particular for at least the elongations to 100% and 120%, the ratio of the laminate has a value which is between 6 and 30, in particular between 8 and 25, in particular between 8 and 20.

In particular, for at least three of the elongations to 40%, 60%, 80%, 100% and 120%, in particular for at least the elongations to 80%, 100% and 120%, the ratio of the laminate has a value which is between 6 and 30, in particular between 8 and 25, in particular between 8 and 20.

In particular, for at least four of the elongations to 40%, 60%, 80%, 100% and 120%, in particular for at least the elongations to 60%, 80%, 100% and 120%, the ratio of the laminate has a value which is between 6 and 30, in particular between 8 and 25, in particular between 8 and 20.

In particular, for the five elongations to 40%, 60%, 80%, 100% and 120%, the ratio of the laminate has a value which is between 6 and 30, in particular between 8 and 25, in particular between 8 and 20.

Preferably, according to a development which itself constitutes an invention independently of the first aspect above, but which can advantageously be implemented in combination therewith, the SETs of the laminate for the elongations to 80%, 100% and 120%, measured during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120%, a return to 0%, a sixth elongation to 40%, a return to 0%, and an elongation to break, are between a lower limit value, for the elongation to 80%, of between 5 and 15, and an upper value, for the elongation to 120%, of between 8 and 30, in particular between 15 and 30, even more particularly between 15 and 25, the SET of the elongations to 80%, 100% and 120% being calculated, respectively, at the fourth, fifth and sixth elongation to 40%.

Preferably, in the range of the elongations of between 40% and 80%, the ratio [SET (as a %)/elongation (as a %/100)] of the laminate is between 6 and 22, in particular between 8 and 17%.

In particular, the SET of the laminate at 40% is between 2.5% and 8%, and/or the SET of the laminate at 60% is between 4% and 15%, in particular between 5% and 10%, and/or the SET of the laminate at 80% is between 5% and 20%, in particular between 10% and 15%, and/or the SET of the laminate at 100% is between 7% and 25%, in particular between 15% and 20%, and/or the SET of the laminate at 120% is between 7% and 30%, in particular between 10% and 27%, in particular between 20% and 25%.

By thus providing a laminate having an increased ratio and/or SET, as described above, during its first use in the usual stretching region of 40% to 80%, preferably 40% to 120% for this type of laminate, in particular when it is mounted on a nappy, an advantage is achieved in that the laminate adjusts automatically, in particular when it is used in a nappy, in particular in the region of the elastic lugs of the waist thereof. The increased SET and/or ratio allows the first user to plastically deform the elastic lug of the nappy and to increase it by stretching it by hand, while maintaining a minimum elasticity in order to adjust it and keep it in place on the nappy wearer.

Preferably, the laminate comprises a second nonwoven that is arranged on the second upper face or lower face, respectively, of the at least one elastic film made of elastomer material.

Preferably, at least one of the values of the ratio [SET (as a %)/elongation (as a %/100)] of the at least one elastic film made of elastomer material,
if breaking of the film occurs beyond the elongation to 120%, measured for the elongations to 40%, 60%, 80%, 100% and 120%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120%, a return to 0%, a sixth elongation to 40%, a return to 0%, and an elongation to break, the SET of the elongations to 40%, 60%, 80%, 100% and 120% being calculated, respectively, at the second, third, fourth, fifth and sixth elongation to 40%, or
if breaking of the film occurs at an elongation of between 100% and 120%, measured for the elongations to 40%, 60%, 80% and 100%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, and an elongation to break, the SET of the elongations to 40%, 60%, 80% and 100% being calculated, respectively, at the second, third, fourth and fifth elongation to 40%, or
if breaking of the film occurs at an elongation of between 80% and 100%, measured for the elongations to 40%, 60% and 80%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, and an elongation to break, the SET of the elongations to 40%, 60% and 80% being calculated, respectively, at the second, third and fourth elongation to 40%, is between 6 and 30, in particular between 8 and 25, in particular between 8 and 20, the elongation being carried out according to a direction in CD (cross direction) or in MD (machine direction).

In particular, for at least two of the elongations to 40%, 60%, 80%, 100% and 120%, in particular for at least the elongations to 100% and 120%, the ratio of the at least one elastic film made of elastomer material has a value which is between 6 and 30, in particular between 8 and 25, in particular between 8 and 20.

In particular, for at least three of the elongations to 40%, 60%, 80%, 100% and 120%, in particular for at least the elongations to 80%, 100% and 120%, the ratio of the at least one elastic film made of elastomer material has a value which is between 6 and 30, in particular between 8 and 25, in particular between 8 and 20.

In particular, for at least four of the elongations to 40%, 60%, 80%, 100% and 120%, in particular for at least the elongations to 60%, 80%, 100% and 120%, the ratio of the at least one elastic film made of elastomer material has a value which is between 6 and 30, in particular between 8 and 25, in particular between 8 and 20.

In particular, for the five elongations to 40%, 60%, 80%, 100% and 120%, the ratio of the at least one elastic film made of elastomer material has a value which is between 6 and 30, in particular between 8 and 25, in particular between 8 and 20.

Preferably, the SETs of the elastic film made of elastomer material, for the elongations to 80%, 100% and 120%, measured during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120%, a return to 0%, a sixth elongation to 40%, a return to 0%, and an elongation to break, the SET of the elongations to 80%, 100% and 120% being calculated, respectively, at the fourth, fifth and sixth elongation to 40%, are between a lower limit value, for the elongation to 80%, of between 5 and 15, and an upper value, for the elongation to 120%, of between 8 and 30, in particular between 15 and 25, the elongation being carried out according to a direction in CD (cross direction) or in MD (machine direction).

Preferably, in the range of elongations of between 40% and 80%, the ratio [SET (as a %)/elongation (as a %/100)] of the at least one elastic film made of elastomer material is between 6 and 22, in particular between 8 and 17.

In particular, the SET of the at least one elastic film made of elastomer material at 40% is between 2.5% and 8%, and/or the SET of the at least one elastic film made of elastomer material at 60% is between 4% and 15%, in particular between 5% and 10%, and/or the SET of the at least one elastic film made of elastomer material at 80% is between 5% and 20%, in particular between 10% and 15%, and/or the SET of the laminate at 100% is between 7% and 25%, in particular 15% and 20%, and/or the SET of the laminate at 120% is between 7% and 30%, in particular between 10% and 27%, in particular between 20% and 25%.

Preferably, for an elongation to 40%, after a prior elongation to 40% and a return to 0%, the SET of the laminate and/or of the at least one elastic film made of elastomer material is greater than 2.25%, in particular greater than 2.5%, 2.75%, 3%, 3.25%, in some cases less than 15%, in particular less than 14%, 13%, 12%, 11%, 10%, 9%, 8%, 7%, 6%.

Preferably, for a third elongation to 40%, after a first elongation to 40%, a return to 0%, a second elongation to 40%, a return to 0%, an elongation to 60% and a return to 0%, the SET of the laminate and/or of the at least one elastic film made of elastomer material is greater than 3.5%, more particularly greater than 4%, in particular greater than 4.5%, 5%, 5.5%, 6%, 6.5%, 7%, in some cases less than 20%, in particular less than 15%, 14%, 13%, 12%, 11%, 10%, 9%.

Preferably, for a fourth elongation to 40%, after a first elongation to 40%, a return to 0%, a second elongation to 40%, a return to 0%, an elongation to 60%, a return to 0%, a third elongation to 40%, a return to 0%, an elongation to 80% and a return to 0%, the SET of the laminate and/or of the at least one elastic film made of elastomer material is greater than 5%, in particular greater than 6%, 7%, 8%, 9%, 10%, in some cases less than 25%, in particular less than 20%, 19%, 18%, 17%, 16%, 15%, 14%, 13%.

Preferably, for a fifth elongation to 40%, after a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100% and a return to 0%, the SET of the laminate and/or of the at least one elastic film made of elastomer material is greater than 6%, in particular greater than 7%, 8%, 9%, 10%, 11%, in some cases less than 30%, in particular less than 25%, 20%, 19%, 18%, 17%.

Preferably, for a sixth elongation to 40%, after a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120% and a return to 0%, the SET of the laminate and/or of the at least one elastic film made of elastomer material is greater than 7%, in particular greater than 8%, 9%, 10%, 11%, 12%, 13%, 14%, 15%, 16%, 17%, 18%, 19%, 20%, in some cases less than 30%, in particular less than 29%, 28%, 27%, 26%, 25%, 24%.

In particular, according to another aspect of the invention itself forming an invention, independently of the other aspects of the invention, but which can also be implemented in a favourable manner in combination with one or more of each of these other aspects, the force at elongation of the laminate in CD at 100% is greater than 7N, in particular greater than 8, 9, 10, 11N.

Preferably, the nonwoven is a nonwoven based on short fibres, in particular a carded nonwoven, for example a spunlace, or a thermobonded carded nonwoven.

Preferably, the thickness of the elastic film made of elastomer material is between 30 and 60 micrometres, in particular between 40 and 60 micrometres, in particular the thickness of the elastic film made of elastomer material is measured in the resting state, before the first elongation. Preferably, an elastic film made of elastomer material is an elastic film that comprises at least 50% by mass of elastomer material, in particular at least 60% by mass of elastomer material, more particularly at least 70% by mass of elastomer material, even more particularly at least 80% by mass of elastomer material, and/or up to 100% by mass of elastomer material, in some cases less than 99% by mass of elastomer material, in particular in some cases less than 98% by mass of elastomer material.

According to a preferred embodiment, fixing of the at least one film to the at least one nonwoven layer is performed by interposition of an adhesive, for example glue, along strips or lines of glue extending lengthwise in the direction MD, and at a distance from one another in the direction CD.

According to a preferred embodiment of the invention, the curve giving the SET of the laminate and/or of the elastic film made of elastomer material as a function of the elongation rises in the range from 40% to 120%, in particular having a slope greater than 0.10 per % of elongation/extension/stretching, in particular between 0.11 and 0.40 per % of elongation/extension/stretching.

The present invention also relates to a nappy for a baby or adult incontinence pants comprising at least one laminate according to the invention, in particular for forming the hook tabs originating laterally from the rear waist of the nappy or incontinence pants, such that the hooks engage with loops originating from the front face of the waist of the nappy, in order to achieve closure of the nappy or incontinence pants.

The present invention also relates to a laminate with hooks, comprising a laminate according to the invention and at least one lap with hooks fixed to the laminate, in particular on the upper nonwoven layer.

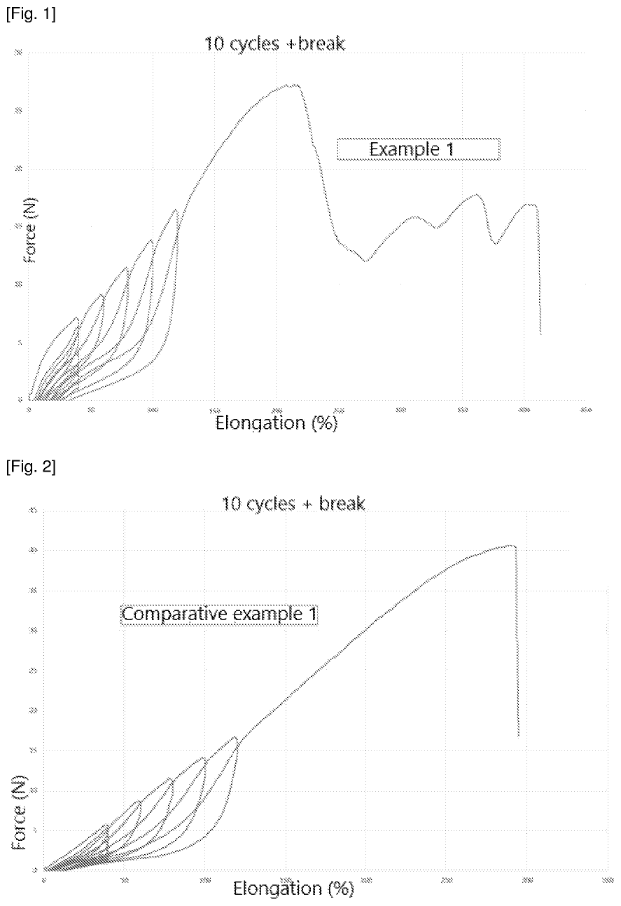

FIG. 1 shows the curves giving the force as a function of the elongation, obtained during the measuring operation of 10 cycles+break for example 1 of a laminate of the present invention.

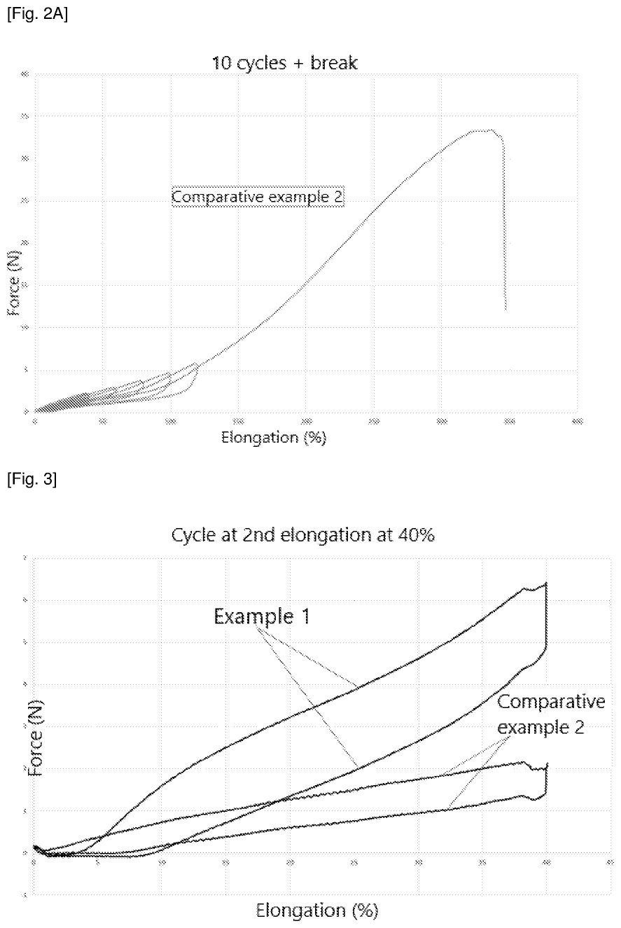

FIG. 2 shows the curves giving the force as a function of the elongation, obtained during the measuring operation of 10 cycles+break for comparative example 1.

FIG. 2A shows the curves giving the force as a function of the elongation, obtained during the measuring operation of 10 cycles+break for comparative example 2.

FIG. 3 shows the parts of the curves of the preceding figures, for example 1 and comparative example 2, respectively, relative to the second stretching/extension/elongation to 40%.

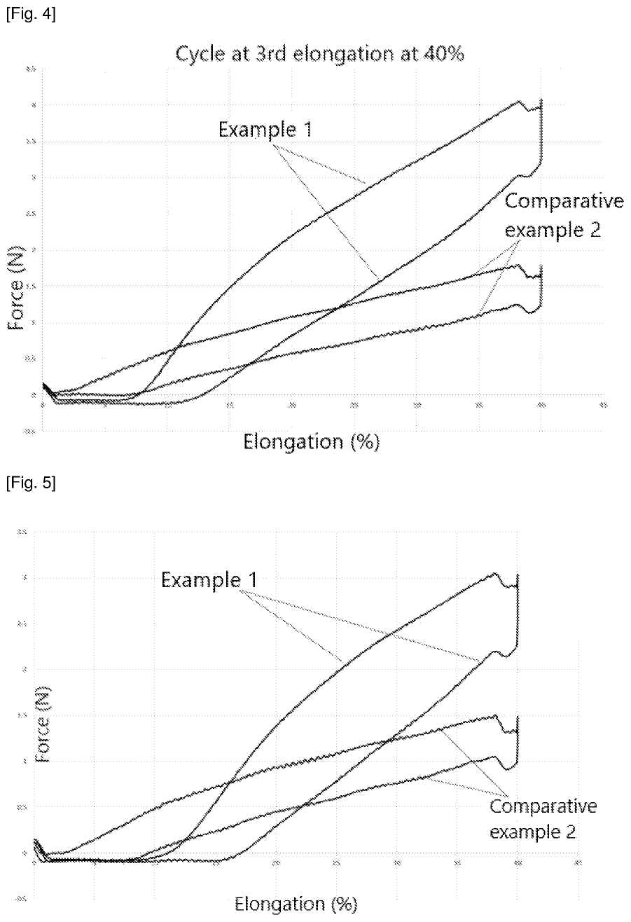

FIG. 4 shows the parts of the curves of the preceding figures, for example 1 and comparative example 2, respectively, relative to the third stretching/extension/elongation to 40%.

FIG. 5 shows the parts of the curves of the preceding figures, for example 1 and comparative example 2, respectively, relative to the fourth stretching/extension/elongation to 40%.

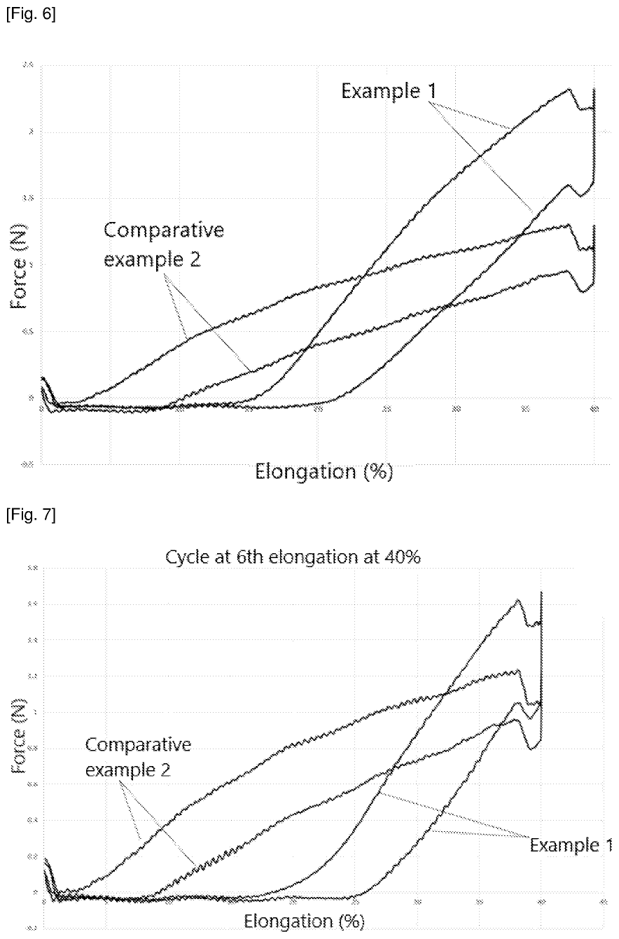

FIG. 6 shows the parts of the curves of the preceding figures, for example 1 and comparative example 2, respectively, relative to the fifth stretching/extension/elongation to 40%.

FIG. 7 shows the parts of the curves of the preceding figures, for example 1 and comparative example 2, respectively, relative to the sixth stretching/extension/elongation to 40%.

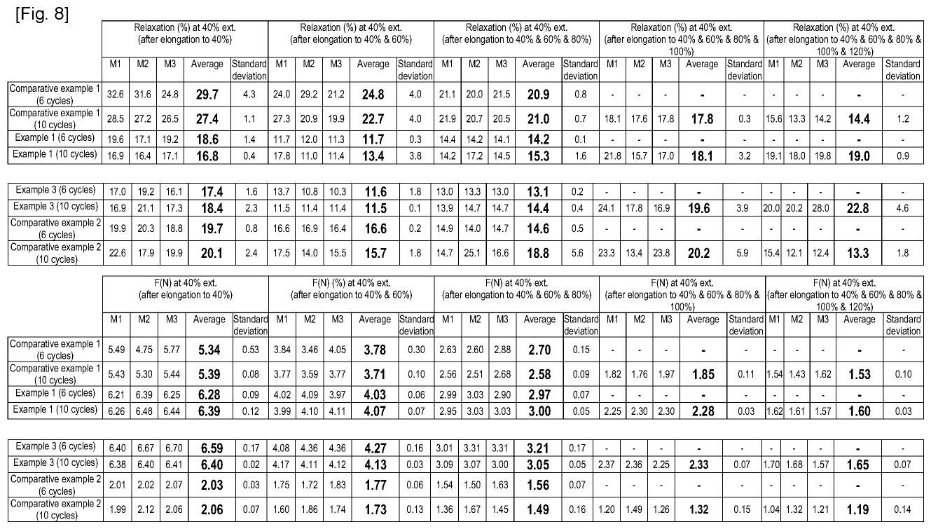

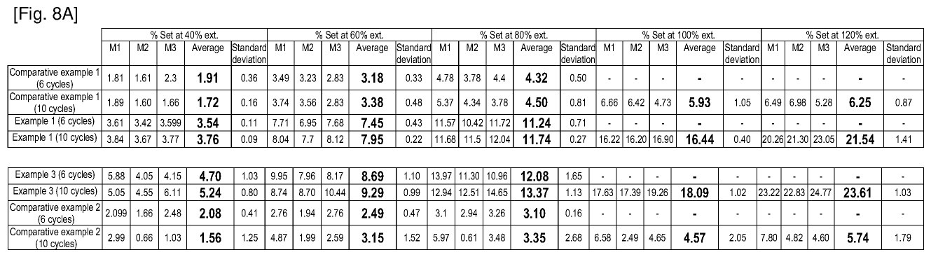

FIG. 8 shows a table giving, for all the samples of Table 1, all the relaxation values (as a percentage) and the forces (in Newtons), as well as their respective averages.

FIG. 8A shows a table giving all the results of the measurements for all the samples, a summary of these measurements being provided in Table 1.

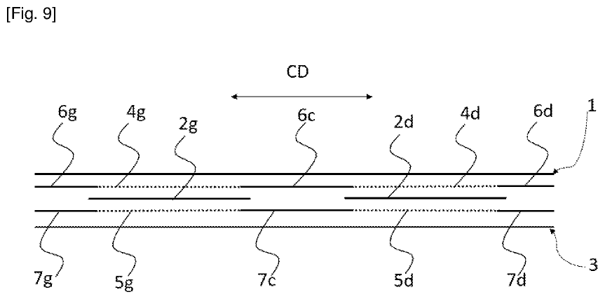

FIG. 9 is a cross-sectional view of a laminate according to the invention.

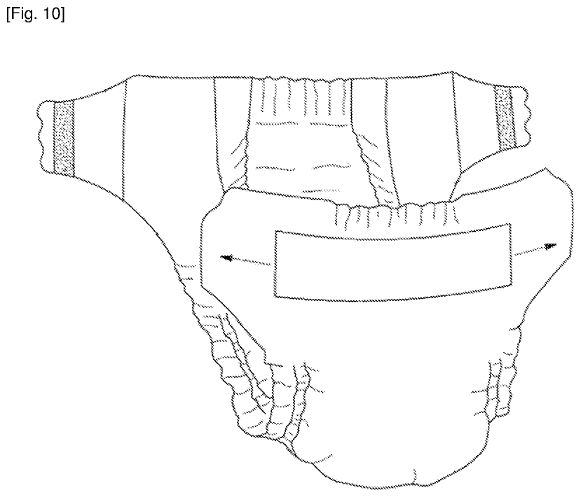

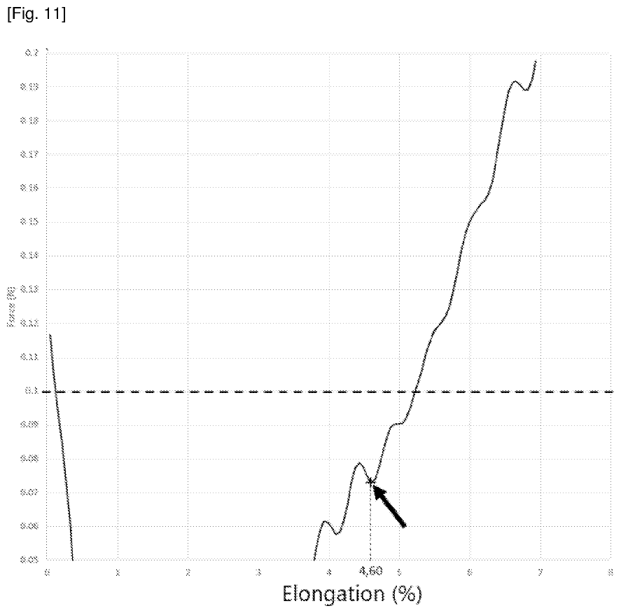

FIG. 10 shows a nappy comprising elastic lugs formed from a laminate according to the invention.

FIG. 11 shows a part of the curve of FIG. 1, illustrating a way of measuring the SET.

In FIG. 9, a laminate comprises, in succession from top to bottom, an upper nonwoven layer 1, two elastic films 2d, 2g made of elastomer material, right-hand and left-hand, respectively, and a lower nonwoven layer 3.

The two elastic films 2d, 2g extend in the direction CD over a smaller distance than the distance over which the two nonwoven layers 1 and 3 extend, such that two edge regions and a central region without elastic film are formed on the left-hand and right-hand edges of the laminate and in the centre.

The upper nonwoven layer 1 and the elastic films 2d, 2g are fixed by interposition, therebetween, of strips or lines 4d, 4g of adhesive, in particular glue, which extend lengthwise in the direction MD and are spaced apart from one another in the direction CD.

In the same way, the lower nonwoven layer 3 and the elastic films 2d, 2g are fixed by interposition, therebetween, of strips or lines 5d, 5g of adhesive, in particular glue, which extend lengthwise in the direction MD and are spaced apart from one another in the direction CD.

In the regions without elastics, the two nonwovens 1 and 3 are fixed directly to one another (i.e. without interposition of elastic film, only the adhesive being present therebetween) by fields 6d, 7d, 6c, 7c, 6g, 7g, respectively right, central and left, extending in the direction MD and having a width that is substantially equal to the respective width of the edge and central regions.

As can be seen from FIG. 9, the two lower and upper nonwoven layers extend, in the direction CD, over the entire width of the laminate. Said width represents the total width of the nonwoven. The two elastic films 2g, 2d made of elastomer material each extend over a respective width that is less than half the width of the laminate. The sum of the two respective widths over which the two films extend represents the total elastic width. Here, the relation or ratio of the total elastic width to the total nonwoven width is between 0.4 and 0.8, and in particular is equal to approximately 0.6 in FIG. 9.

According to the invention, it is possible to use a glue such as non-reactive hot melt glues, for example H2511 by Bostik, or XPU18314 by Bostik (reactive PU glue). Preferably, these glues will have a chemical nature similar to the elastomer film. For example, if one of these glues is analysed using an infrared spectrometer, in order to identify the chemical functions, or using liquid chromatography to separate and quantify the substances, preferably traces of one or more components or their derivatives, of the material or materials of the elastomer film, will be identified.

Preferably, these glues are based on SIS, SBS, SEBS and SEPS, allowing for good affinity with the film due to the similar chemical materials.

Preferably, the glue layer has a basis weight of less than 15 g/m², in particular less than 12 g/m², more preferably less than 8 g/m².

In the present invention, a nonwoven is intended to mean a product obtained following formation of a lap of fibres and/or filaments which has been consolidated. The consolidation may be mechanical, chemical or thermal, and results in the presence of bonds between the fibres and/or the filaments. This consolidation may be direct, i.e. performed directly between the fibres and/or the filaments by means of welding, or it may be indirect, i.e. by means of an intermediate layer between the fibres and/or the filaments, for example a layer of glue or a layer of binder. The term “nonwoven” relates to a structure in the form of a tape or lap of fibres and/or filaments which are interlaced in a non-uniform or irregular manner, or at random. A nonwoven may have a single layer structure or a structure comprising a plurality of layers. A nonwoven may be made from different synthetic and/or natural materials. The natural materials, by way of example, are cellulose fibres such as cotton, jute, paper pulp, linen or the like, and may also include recycled cellulose fibres, such as rayon or viscose (cellulose acetate). The natural fibres for a nonwoven material may be prepared by using various processes, such as carding. Synthetic materials, by way of example but without restriction thereto, include synthetic thermoplastic polymers, which are known to form fibres and/or filaments which include, without restriction thereto, polyolefins, for example polyethylene, polypropylene, polybutylene, and the like; polyamide, for example polyamide 6, polyamide 6.6, polyamide 10, polyamide 11, polyamide 12, and the like; polyesters, for example polyethylene terephthalates, polybutylene terephthalates, polylactic acids (PLA) or PHA (polyhydroxyalkanoates), and the like, polycarbonates, polystyrenes, thermoplastic elastomers, vinyl polymers, polyurethanes, and mixtures and co-polymers thereof. Some of these materials may be bioplastic, for example bio-sourced (for example bio-PE, PLA, polyamide 11, viscose (cellulose acetate), and the like) and/or biodegradable (PLA and the like). In a general manner, fibres and filaments differ mainly in their length and their manufacturing method.

Continuous filaments refer to the individual elements, which are very long compared with the diameter of their cross-section, extruded in a continuous manner in order to directly form a nonwoven lap, which can then be consolidated by thermobonding or any other means making it possible to achieve the desired performance, and/or transport thereof. Preferably, the continuous filaments have a length greater than 120 mm.

Fibre is meant as the generic term for denoting a textile material or a textile material element of reduced length, less than the length of the continuous filaments, and able to be spun and/or used in the formation of nonwovens. Two types of fibres are distinguished, short fibres formed in a discontinuous manner with a short length less than 50 mm (preferably from 25 mm to 50 mm), and long fibres formed in a discontinuous manner and with a long length greater than 50 mm (preferably from 60 mm to 120 mm).

Unlike continuous filaments which are consolidated directly after extrusion, the fibres are usually oriented and organised in a lap during a carding step that is well known to a person skilled in the art. This lap can then be consolidated by thermobonding or any other means making it possible to achieve the desired performance, and/or transport thereof.

According to the present invention, film means a material of a sheet or wrap type, the length and the width of which are each greater than the thickness (for example in a ratio of 10', 50×, or even 1000× or more). Typically, a film has a thickness of less than 0.7 mm, in particular less than 0.5 mm or thinner. In particular, a string or a thread or a set of strings and/or threads are not films.

Regarding the elastic films, it is possible to use a thermoplastic elastomer material or a mixture comprising a material of this kind, in particular recyclable and of the polyolefin elastomer type (for example based on polypropylene (PP) or polyethylene (PE), for example the Vistamaxx range by Exxon, or the Infuse, Engage and Versify ranges by Dow Chemical, and which can be associated with one another or with other polyolefins, depending on the performance intended for the application. It is possible to use, for example, a propylene elastomer, in particular the “Vistamaxx” range by EXXON MOBIL under the references 6000 and/or 6102 and/or 6102FL and/or 6202 and/or 6202FL and/or 6502, 7050BF and/or 7810, and/or an olefin block copolymer, in particular the “Infuse” range by DOW CHEMICAL under the references 9000 and/or 9007 and/or 9010 and/or 9077 and/or 9100 and/or 9107, and/or a polyolefin elastomer, in particular the “Engage” range by DOW CHEMICAL under the references 8402 and/or 8401 and/or 8411 and/or 8407 and/or 8137 and/or 8200 and/or 8207, and/or a propylene/ethylene copolymer, in particular the “Versify” range by DOW CHEMICAL under the references 3200 and/or 3300 and/or 3401 and/or 4200, and/or a thermoplastic elastomer (TPE-E) (copolyesters), for example APINAT DP1888-75CV or APINAT DP1888-75 by TRINSEO and/or in particular NP-EL 208-65 by NaturPlast, and/or a thermoplastic urethane elastomer (TPE-U) and/or thermoplastic amide elastomer (TPE-A), thermoplastic styrene elastomer (TPE-S) and/or, more broadly, polyesters having a hardness of less than 90 Shore A, in particular between 50 and 90 Shore A.

It is in particular possible to manufacture the laminate according to the invention as described in EP-A-1783257, by preparing the elastic film or films by extruding it or them from one or more extrusion dies in order to obtain, after cooling, one or more substantially solid films, by laminating the substantially solid film or films onto a nonwoven layer by interposing a fixing agent, and then, in the case of nonwovens that are not elastic or are insufficiently elastic, by stretching the laminate transversely in order to break the fibres of the nonwoven(s) and/or the cohesion therebetween. Said transverse stretching can in particular be carried out by “activation”, i.e. passing the composite, originating from the lamination, into an activation point in which two toothed rollers ensure transverse stretching of the laminate in order to “activate” the laminate, i.e. increase its stretching capacity by “stretching” the nonwovens that are not elastic or only slightly elastic, and/or the elastic films, which releases the elasticity of the elastics in the laminate and thus increases the elasticity thereof.

### Comparative Example 1

Comparative example 1 comprises an elastic film made of elastomer material that is fixed between two layers of nonwoven via two layers of glue. The upper nonwoven is a nonwoven spunlace, designated by the reference “Sawasoft”, having a basis weight of 30 g/m², available from the Sandler company. The lower nonwoven is a thermobonded carded nonwoven, designated by the reference “Sawabond 4111”, having a basis weight of 22 g/m², available from the Sandler company. The elastic film made of elastomer material originates from an SIS material having a basis weight of 50 g/m². The glue, based on SIS, has a basis weight of approximately 4.5 g/m² per layer of glue. The product is activated according to a disc activation block, typically used in the field.

### Comparative Example 2

Comparative example 2 comprises an elastic film made of elastomer material that is fixed between two layers of nonwoven via two layers of glue. The upper nonwoven, identical to the lower nonwoven, is a nonwoven spunlace, with the reference “Sawasoft 2626”, having a basis weight of 25 g/m², available from the Sandler company. The elastic film made of elastomer material originates from an SIS material having a basis weight of 50 g/m². The glue, based on SIS, has a basis weight of approximately 4.5 g/m² per layer of glue. The product is not activated.

### Example 1

Example 1 comprises an elastic film made of elastomer material that is fixed between two layers of nonwoven via two layers of glue. The upper nonwoven, identical to the lower nonwoven, is a nonwoven spunlace 100% PLA-FLAT, with the reference “15.020.001.0200.01.02”, having a basis weight of 20 g/m², available from the JACOB HOLM INDUSTRIES company. The elastic film made of elastomer material originates from a TPE-E with reference “APINAT DP1888-75CV” available from the TRINSEO company and extruded at a basis weight of 60 g/m². The glue has an average basis weight of approximately 4 g/m² per layer of glue and has reference “XPU18314” and available from the BOSTIK company. The glue is applied in full in the zones without elastic and in the elastic zone close to the ends of the elastic, and in the form of lines or strips in the other zones. The laminate is passed into an activation block made up of two rollers comprising stacked discs, the teeth of which interpenetrate by 3.33 mm without the product being held laterally, for example by central and/or lateral straps.

### Example 3

Example 3 is similar to example 1 but with the following differences: the average basis weight of the glue is approximately 3.5 g/m², and the glue is distributed differently, i.e. the lines of glue and/or strips of glue are spaced further apart from one another, in CD, in the elastic zone.

In order to measure the SET, in particular at 100%, and the ratio [SET (as a %)/elongation (as a %/100)] over the elongation of an element such as a laminate or an elastic film, it is possible to proceed as follows, using a dynamometer, for example “Zwick/Roell Z2.5 dynamometer” type 2.5 Kn Zwicki with “testXpert III” application software and a point acquisition frequency of 500 Hz, a 100 N load cell, and for example upper and lower jaws according to the “8297-2.5 kN” model.

### Preparation of the Sample:

Step 0a: The element, for example an elastic laminate or an elastic film made of elastomer material, for example a laminate originating directly from a reel or a nappy from a packet of nappies, and in a normal atmosphere, temperature 23° C. (+/−2° C.) and at a relative humidity of 50% (+/−5%), is conditioned for a duration of 24 h.

Step 0b: A sample having dimensions of 40 mm in width and at least 40 mm in height is cut, preferably using a circular-blade cutter, such that it can be characterised over a height of 30 mm and a width of 40 mm. Said sample is cut from the laminate to ensure that it comprises the elastic film made of elastomer material which extends over its entire width and its entire length.

The product is stretched to the elongation value at which the SET is to be measured, in particular to 100%, for example at a speed of 508 mm/min, by vertical displacement of the upper jaw, the lower jaw being fixed, and then it is held in the position for 30 seconds and then returned to the initial position at a constant speed, where it is left for 60 seconds (end of first cycle), and then it is stretched again to 100%, held for 30 seconds, and returned to the initial position (end of second cycle). Thus, the curve giving the stretching force as a function of the elongation as a % is obtained, said curve having a hysteresis which makes it possible to determine the SET by the following calculation formula:

SET=L1−L0
where:
L0: Intersection point with the x-axis (elongation as a %) at the start of the test, i.e. the beginning of the first cycle.
L1: Intersection point with the x-axis (elongation as a %) at the start of the second cycle, after returning to the original position and waiting 60 seconds.
   The method described above can also be implemented at an elongation value other than 100%, for example a value of 40%, 60%, 80% or 120%, provided that the elongation value used, at which the SET is to be measured, is not less than a prior elongation of the product/sample.

Calculation of the SET and of the ratio of the SET over the extension (or elongation) (as a %/100) for the values of 40%, 60%, 80%, 100% and 120%
1. The inter-jaw distance is adjusted to 30 mm,
2. the cut sample is positioned in the jaws in such a way that the 40 mm width is received in full in the jaws, and 30 mm height of elastic laminate is tested,
3. a preload up to 0.05 N is applied,
4. the measurements are performed (the measurement results are in the form of a curve giving the force applied as a function of the elongation, or are derived from said curve) during an elongation at a speed of 508 mm/min up to 40% elongation without delay (first elongation/extension/stretching to 40%),
5. the initial inter-jaw position of 30 mm is returned to, and the sample is held for 60 seconds,
6. the measurements are performed during an elongation at a speed of 508 mm/min up to 40% elongation, and the sample is held for 30 seconds (second elongation/extension/stretching to 40%),
7. the initial inter-jaw position of 30 mm is returned to, and the sample is held for 60 seconds,
8. the measurements are performed during an elongation at a speed of 508 mm/min up to 60% elongation without delay,
9. the initial inter-jaw position of 30 mm is returned to, and the sample is held for 60 seconds,
10. the measurements are performed during an elongation at a speed of 508 mm/min up to 40% elongation, and the sample is held for 30 seconds (third elongation/extension/stretching to 40%),
11. the initial inter-jaw position of 30 mm is returned to, and the sample is held for 60 seconds,
12. the measurements are performed during an elongation at a speed of 508 mm/min up to 80% elongation without delay,
13. the initial inter-jaw position of 30 mm is returned to, and the sample is held for 60 seconds,
14. the measurements are performed during an elongation at a speed of 508 mm/min up to 40% elongation, and the sample is held for 30 seconds (fourth elongation/extension/stretching to 40%),
15. the initial position of 30 mm is returned to, and the sample is held for 60 seconds,
16. the measurements are performed during an elongation at a speed of 508 mm/min up to 100% elongation without delay,
17. the initial inter-jaw position of 30 mm is returned to, and the sample is held for 60 seconds,
18. the measurements are performed during an elongation at a speed of 508 mm/min up to 40% elongation, and the sample is held for 30 seconds (fifth elongation/extension/stretching to 40%),
19. the initial position of 30 mm is returned to, and the sample is held for 60 seconds,
20. the measurements are performed during an elongation at a speed of 508 mm/min up to 120% elongation without tempo,
21. the initial inter-jaw position of 30 mm is returned to, and the sample is held for 60 seconds,
22. the measurements are performed during an elongation at a speed of 508 mm/min up to 40% elongation, and the sample is held for 30 seconds (sixth elongation/extension/stretching to 40%),
23. the initial position of 30 mm is returned to, and the sample is held for 60 seconds, and then
24. the measurements are performed during an elongation at a speed of 508 mm/min up to breakage of the elastic laminate.

Thus, at the end, a curve (10 cycles+break) is obtained like that obtained for the examples and comparative examples described below and shown in FIG. 1. The SETs are then measured, on the curve (10 cycles+break) for each elongation value, respectively to 40%, 60%, 80%, 100% and 120% and these are put into Table 1 below. FIG. 11 shows the curve giving the force as a function of the percentage elongation. The SET is taken directly from here and corresponds to the value of the elongation for a selected point of the curve in the force interval between [0.05 N-0.1 N], said point being selected proceeding from the point having the greatest elongation and decreasing strictly until the point having the lowest force is reached. Thus, in FIG. 11, the SET recorded is 4.60%.

It is possible to omit steps 16 to 23, corresponding to elongation to 100 and 120%, in particular when breakage of the sample occurs before these elongation values, in order to obtain, at the end, a curve referred to as 6 cycles+break or 8 cycles+break, like that obtained for the examples and comparative examples described below. The SETs are then measured, on the curve (6 cycles+break) for each elongation value, respectively to 40%, 60% and 80%. FIG. 8 also shows, for each example and comparative example, each of the second, third, fourth, fifth and sixth elongations to 40%, the relaxation values (as a percentage) obtained and their averages, as well as the forces (in Newtons) obtained and their averages. Table 1 below shows the results obtained for comparative examples 1 and 2, and examples 1 and 3 above, for the 6 cycles+break and 10 cycles+break tests. For these measurements, three samples, M1, M2 and M3, were made for each of the examples and comparative examples, and the average of the three was entered. The results for all the samples are set out in the table in FIG. 8.

| | SET & RATIO SET (%)/elongation (%/100) | | | | | | | | | |
|---|---|---|---|---|---|---|---|---|---|---|
| | 40% | | 60% | | 80% | | 100% | | 120% | |
| | SET | | SET | | SET | | SET | | SET | |
| | (%) | Ratio | (%) | Ratio | (%) | Ratio | (%) | Ratio | (%) | Ratio |
| Comparative | 1.91 | 4.77 | 3.18 | 5.31 | 4.32 | 5.40 | — | — | — | — |
| example 1 | | | | | | | | | | |
| (6 cycles) | | | | | | | | | | |
| Comparative | 1.72 | 4.29 | 3.38 | 5.63 | 4.50 | 5.62 | 5.93 | 5.93 | 6.25 | 5.21 |
| example 1 | | | | | | | | | | |
| (10 cycles) | | | | | | | | | | |
| Example 1 | 3.54 | 8.86 | 7.45 | 12.41 | 11.24 | 14.05 | — | — | — | — |
| (6 cycles) | | | | | | | | | | |
| Example 1 | 3.76 | 9.40 | 7.95 | 13.26 | 11.74 | 14.68 | 16.44 | 16.44 | 21.54 | 17.95 |
| (10 cycles) | | | | | | | | | | |
| Example 3 | 4.70 | 11.74 | 8.69 | 14.49 | 12.08 | 15.10 | — | — | — | — |
| (6 cycles) | | | | | | | | | | |
| Example 3 | 5.24 | 13.09 | 9.29 | 15.49 | 13.37 | 16.71 | 18.09 | 18.09 | 23.61 | 19.67 |
| (10 cycles) | | | | | | | | | | |
| Comparative | 2.08 | 5.20 | 2.49 | 4.14 | 3.10 | 3.88 | — | — | — | — |
| example 2 | | | | | | | | | | |
| (6 cycles) | | | | | | | | | | |
| Comparative | 1.56 | 3.90 | 3.15 | 5.25 | 3.35 | 4.19 | 4.57 | 4.57 | 5.74 | 4.78 |
| example 2 | | | | | | | | | | |
| (10 cycles) | | | | | | | | | | |

1. A laminate extending widthwise, in particular in the direction CD, and lengthwise, particularly in the direction MD, comprising:
at least one nonwoven layer, said nonwoven layer or layers forming the at least one nonwoven layer extending over a total nonwoven width, in particular in the direction CD, and
at least one elastic film made of elastomer material fixed by an upper face or lower face, respectively, to a lower face or upper face, respectively, of the at least one nonwoven layer, the elastic film or films made of elastomer material forming the at least one elastic film made of elastomer material extending over a total elastic width, characterised in that
the ratio equal to the total elastic width to the total nonwoven width is between 0.3 and 0.9; and
the SET at 100% elongation of the laminate and/or of the at least one elastic film made of elastomer material, measured, in the width direction, with a sample having a length and a width and wherein the elastomer material extends along the entire length and across the entire width of the sample, is greater than 6%, in particular between 6% and 30%.

2. The laminate according to claim 1, characterised in that the ratio equal to the total elastic width to the total nonwoven width is between 0.4 and 0.8.

3. The laminate according to either claim 1, characterised in that the SET at 100% elongation of the laminate, measured with a sample having a length and a width and wherein the elastic extends along the entire length and across the entire width of the sample, is greater than 15%, in particular between 15% and 30%.

4. The laminate according to claim 1, characterised in that the material of the elastic film or films made of an elastomer material has a Shore A hardness of between 50 and 90.

5. The laminate according to claim 4, characterised in that the material of the elastic film or films made of elastomer material has a Shore A hardness of between 60 and 80.

6. The laminate according to claim 1, characterised in that at least one of the values of the ratio [SET (as a %)/elongation (as a %/100)] of the laminate,
if breaking of the laminate occurs beyond the elongation to 120%, measured for the elongations to 40%, 60%, 80%, 100% and 120%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120%, a return to 0%, a sixth elongation to 40%, a return to 0%, and an elongation to break, the SET of the elongations to 40%, 60%, 80%, 100% and 120% being calculated, respectively, at the second, third, fourth, fifth and sixth elongation to 40%, or
if breaking of the laminate occurs at an elongation of between 100% and 120%, measured for the elongations to 40%, 60%, 80% and 100%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, and an elongation to break, the SET of the elongations to 40%, 60%, 80% and 100% being calculated, respectively, at the second, third, fourth and fifth elongation to 40%, or
if breaking of the laminate occurs at an elongation of between 80% and 100%, measured for the elongations to 40%, 60% and 80%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, and an elongation to break, the SET of the elongations to 40%, 60% and 80% being calculated, respectively, at the second, third and fourth elongation to 40%,
is between 6 and 30, in particular between 8 and 25, in particular between 8 and 20, the elongation being carried out according to a direction in CD (cross direction) or in MD (machine direction).

7. The laminate according to claim 6, characterised in that, for at least two of the elongations to 40%, 60%, 80%, 100% and 120%, in particular for at least the elongations to 100% and 120%, the ratio of the laminate has a value which is between 6 and 30, in particular between 8 and 25, in particular between 8 and 20.

8. (canceled)

9. (canceled)

10. (canceled)

11. The laminate according to claim 1, characterised in that the SETs of the laminate for the elongations to 80%, 100% and 120%, measured during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120%, a return to 0%, a sixth elongation to 40%, a return to 0%, and an elongation to break, are between a lower limit value, for the elongation to 80%, of between 5 and 15, and an upper value, for the elongation to 120%, of between 8 and 30, in particular between 15 and 25, the SET of the elongations to 80%, 100% and 120% being calculated, respectively, at the fourth, fifth and sixth elongation to 40%.

12. (canceled)

13. (canceled)

14. The laminate according to claim 1, characterised in that the laminate comprises a second nonwoven that is arranged on the second upper face or lower face, respectively, of the elastic film made of elastomer material.

15. The laminate according to claim 1, characterised in that one of the values of the ratio [SET (as a %)/elongation (as a %/100)] of the at least one elastic film made of elastomer material,
if breaking of the film occurs beyond the elongation to 120%, measured for the elongations to 40%, 60%, 80%, 100% and 120%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120%, a return to 0%, a sixth elongation to 40%, a return to 0%, and an elongation to break, the SET of the elongations to 40%, 60%, 80%, 100% and 120% being calculated, respectively, at the second, third, fourth, fifth and sixth elongation to 40%, or
if breaking of the film occurs at an elongation of between 100% and 120%, measured for the elongations to 40%, 60%, 80% and 100%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, and an elongation to break, the SET of the elongations to 40%, 60%, 80% and 100% being calculated, respectively, at the second, third, fourth and fifth elongation to 40%, or
if breaking of the film occurs at an elongation of between 80% and 100%, measured for the elongations to 40%, 60% and 80%, during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, and an elongation to break, the SET of the elongations to 40%, 60% and 80% being calculated, respectively, at the second, third and fourth elongation to 40%,
is between 6 and 30, in particular between 8 and 25, in particular between 8 and 20, the elongation being carried out according to a direction in CD (cross direction) or in MD (machine direction).

16. (canceled)

17. (canceled)

18. (canceled)

19. The laminate according to claim 15, characterised in that, for the five elongations to 40%, 60%, 80%, 100% and 120%, the ratio of the at least one elastic film made of elastomer material has a value which is between 6 and 30, in particular between 8 and 25, in particular between 8 and 20.

20. The laminate according to claim 15, characterised in that the SETs of the elastic film made of elastomer material, for the elongations to 80%, 100% and 120%, measured during a measuring operation in which, in succession, the following are carried out: a first elongation to 40%, a return to 0%, a second elongation to 40%, a return to 0%, an elongation to 60%, a return to 0%, a third elongation to 40%, a return to 0%, an elongation to 80%, a return to 0%, a fourth elongation to 40%, a return to 0%, an elongation to 100%, a return to 0%, a fifth elongation to 40%, a return to 0%, an elongation to 120%, a return to 0%, a sixth elongation to 40%, a return to 0%, and an elongation to break, the SET of the elongations to 80%, 100% and 120% being calculated, respectively, at the fourth, fifth and sixth elongation to 40%, are between a lower limit value, for the elongation to 80%, of between 5 and 15, and an upper value, for the elongation to 120%, of between 8 and 30, in particular between 15 and 25, the elongation being carried out according to a direction in CD (cross direction) or in MD (machine direction).

21. (canceled)

22. (canceled)

23. (canceled)

24. (canceled)

25. (canceled)

26. (canceled)

27. (canceled)

28. The laminate according to claim 1, characterised in that the force at elongation of the laminate in CD at 100% is greater than 7 N.

29. The laminate according to claim 1, characterised in that the nonwoven is a nonwoven based on short fibres, in particular a carded nonwoven, for example a spunlace, or a thermobonded carded nonwoven.

30. The laminate according to claim 1, characterised in that fixing of the at least one film to the at least one nonwoven layer is performed by interposition of an adhesive, for example glue, along strips or lines of glue extending lengthwise in the direction MD, and at a distance from one another in the direction CD.

31. A laminate with hooks, comprising a laminate according to claim 1 and at least one lap with hooks fixed to the laminate, in particular on the upper nonwoven layer.

32. Nappy A nappy for a baby or adult incontinence pants comprising at least one laminate according to claim 1, for forming hook tabs originating laterally from the rear waist of the nappy or incontinence pants, such that hooks engage with loops originating from the front face of the waist of the nappy, in order to achieve closure of the nappy or incontinence pants.

33. A nappy for a baby or adult incontinence pants comprising at least one laminate according to claim 31, for forming hook tabs originating laterally from the rear waist of the nappy or incontinence pants, such that hooks engage with loops originating from the front face of the waist of the nappy, in order to achieve closure of the nappy or incontinence pants.
